(19) United States
(12) Patent Application Publication
(10) Pub. No.: US 2023/0255543 A1
(43) Pub. Date: Aug. 17, 2023

(54) SENSOR

(71) Applicant: Grace Imaging Inc., Shinjuku-ku, Tokyo (JP)

(72) Inventors: Daisuke NAKASHIMA, Tokyo (JP); Masayasu FUJIOKA, Tokyo (JP)

(21) Appl. No.: 18/013,218

(22) PCT Filed: Jun. 29, 2021
(86) PCT No.: PCT/JP2021/024627

§ 371 Date: Dec. 27, 2022

(30) Foreign Application Priority Data
Jun. 29, 2020 (JP) 2020-111364

(51) Int. Cl. A61B 5/00

(52) U.S. Cl. CPC A61B 5/4266; A61B 5/6832

(57) ABSTRACT

Provided is a sensor capable of suppressing deviation in the state where the sensor is in contact with a living body. A sensor 1 converts, to an electrical signal, the amount of a substance to be measured contained in a secretion from a living body H, the sensor 1 comprising: a sensor main body 10 having an electrode part 11 that is disposed on the contact surface side contacting the living body H and a substrate 12 that is disposed on the exposed surface side opposite to the contact surface; and a fixing part 20 for fixing the sensor main body 10 to the living body H, wherein the fixing part 20 has a flexible sheet 21 that is attached to the living body H and fixes the sensor main body 10 to the living body H, and a spacer part 22 that is disposed between the flexible sheet 21 and the exposed surface and has a thickness equal to or thicker than that of the sensor main body 10.

### NOTICE OF COPYRIGHTS AND TRADE DRESS

A portion of the disclosure of this patent document contains material which is subject to copyright protection. This patent document may show and/or describe matter which is or may become trade dress of the owner. The copyright and trade dress owner has no objection to the facsimile reproduction by anyone of the patent disclosure as it appears in the Patent and Trademark Office patent files or records, but otherwise reserves all copyright and trade dress rights whatsoever.

### RELATED APPLICATION INFORMATION

This patent claims priority from International PCT Patent Application No. PCT/JP2021/024627, filed Jun. 29, 2021 entitled, “SENSOR”, which claims priority to Japanese Patent Application No. 2020-111364, filed Jun. 29, 2020, all of which are incorporated herein by reference in their entirety.

### TECHNICAL FIELD

The present invention relates to a sensor.

### BACKGROUND

Conventionally, there is known a sensor that measures a measurement target contained in a secretion from a living body. The sensor includes an absorption layer capable of absorbing the secretion, an enzyme layer that contains an enzyme, and an electrode part. The sensor detects, by the electrode part, an electrode-detectable substance generated from the measurement target substance under presence of the enzyme (see Non-Patent Documents 1 and 2).
Non-Patent Document 1: Wei Gao et al. (Nature, Vol.529,509-(2016))
Non-Patent Document 2: Wenzhao Jia et al. (Anal. Chem., Vol.85, 6553-6560(2013)).

### DISCLOSURE OF THE INVENTION

### Problems to Be Solved by the Invention

Incidentally, the sensor is positioned at a prescribed position of a living body to be in contact therewith. In continuous measurements, the measurements may become unstable when the sensor moves from the contact position. Therefore, at the time of measurements, it is preferable for the sensor to be suppressed from being deviated while being in contact with the contact position.

The present invention is designed in view of the foregoing issue, and it is an object thereof to provide a sensor capable of suppressing deviation while being in contact with a living body.

### Means for Solving the Problems

The present invention relates to a sensor converting an amount of a measurement target substance contained in a secretion from a living body into an electrical signal, the sensor including: a sensor main body including an electrode part disposed on a contact surface side contacting the living body, and a substrate disposed on an exposed surface side opposite to the contact surface; and a fixing part that fixes the sensor main body to the living body, in which the fixing part includes: a flexible sheet that is attached to the living body, and fixes the sensor main body to the living body; and a spacer part disposed between the flexible sheet and the exposed surface, the spacer part having a thickness equal to or thicker than a thickness of the sensor main body.

Furthermore, it is preferable that the flexible sheet include an adhesive layer on a surface contacting the living body, and the spacer part be attached to the flexible sheet by the adhesive layer.

It is also preferable that the flexible sheet have an area larger than a contact surface with the spacer part.

Furthermore, it is preferable that the sensor further include a cover part that covers the fixing part, the cover part pressing the fixing part against the living body.

Moreover, it is preferable that the sensor main body contact a forehead, an upper arm, a back, or a wrist of a human body.

Furthermore, it is preferable that the electrode part include: an absorption layer capable of absorbing the secretion; an enzyme layer containing an enzyme that reacts with the absorbed secretion; and an electrode layer that converts a reaction with the secretion into an electrical signal.

It is also preferable that the enzyme layer contain at least one kind selected from lactate oxidase, glucose oxidase, and alcohol oxidase.

Furthermore, it is preferable that the absorption layer contain a polymer material having a chemically bonded crosslinked structure, and the absorption layer cover end faces of the enzyme layer and the electrode layer.

### Effects of the Invention

According to the present invention, it is possible to provide the sensor capable of suppressing deviation while being in contact with the living body.

### BRIEF DESCRIPTION OF THE DRAWINGS

FIG. 1 is a schematic plan view illustrating a sensor according to an embodiment of the present invention;

FIG. 2 is a sectional view taken along a line A-A in FIG. 1; and

FIG. 3 is a sectional view illustrating a sensor main body of the sensor according to the embodiment.

### PREFERRED MODE FOR CARRYING OUT THE INVENTION

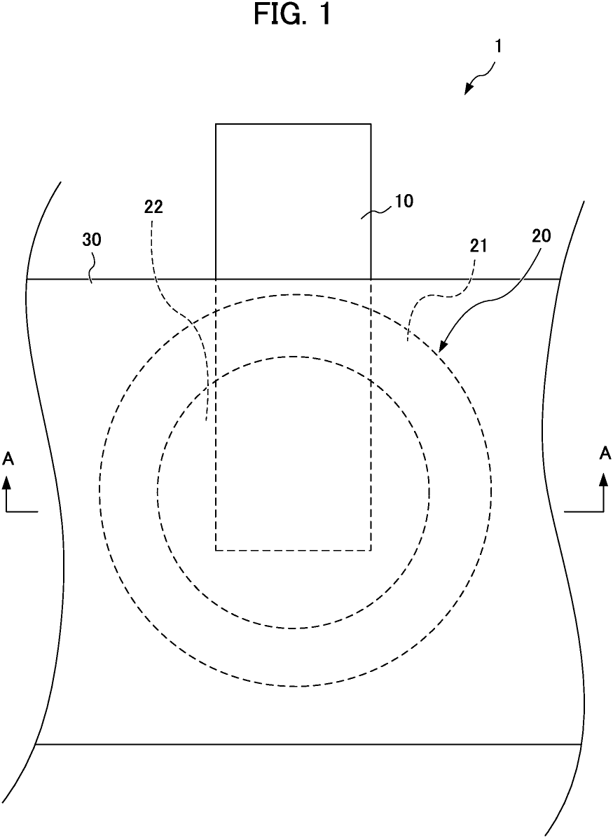

Hereinafter, a sensor 1 according to an embodiment of the present invention will be described with reference to FIG. 1 to FIG. 3. First, an overview of the sensor 1 will be described. The sensor 1 is for measuring a measurement target contained in a secretion from a living body, for example. The sensor 1 absorbs the secretion, and generates an electrode-detectable substance from the measurement target contained in the absorbed secretion. The sensor 1 measures an electric current value generated due to an electrochemical reaction of the generated electrode-detectable substance to electrically detect the density of the electrode-detectable substance. With such a configuration, the sensor 1 electrically detects the density of the measurement target substance contained in the secretion from a living body H.

At the time of measurement, the sensor 1 is fixed for the position of the living body H where the measurement target is to be measured. By being fixed while being in contact with the living body H, the sensor 1 can measure the secretion continuously in terms of time. In the following embodiment, the sensor 1 is maintained to be in a state of being fixed at the position of the living body H where the measurement target is to be measured such that the secretion at a prescribed position of the living body H can be appropriately absorbed.

Note that the sensor 1 measures the measurement targets contained in sweat, tears, saliva, urine, and the like, for example, as the secretions from the living body H. As the measurement targets, the sensor 1 measures lactic acid, glucose (grape sugar), ethanol, uric acid, urea, vitamins, free cholesterol, phosphate ions, and the like, for example. Among those secretions, sweat is preferable in terms of the fixing method and the position to be in contact with the living body.

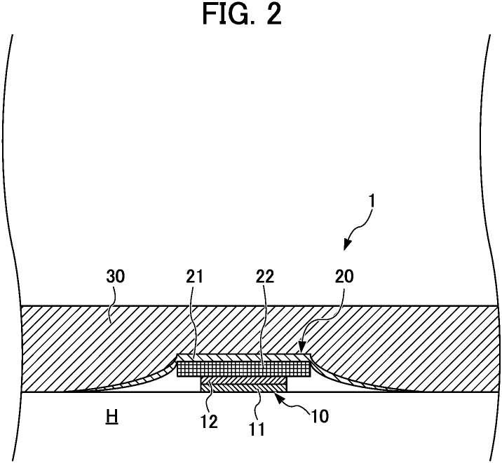

Next, the configuration of the sensor 1 according to the embodiment will be described. The sensor 1 is a sensor 1 that converts the amount of the measurement target substance contained in the secretion from the living body H into an electrical signal. The sensor 1 electrically converts the amount of the measurement target substance contained in the secretion from a person or a horse as the living body H, for example. As an example, the sensor 1 is fixed by being brought into contact with the forehead, an upper arm, the back, or a wrist of a human body. The sensor 1 includes a sensor main body 10, a fixing part 20, and a cover part 30 as illustrated in FIG. 1 and FIG. 2.

As illustrated in FIG. 2, the sensor main body 10 is a device that actually comes in contact with the living body H to measure the amount of the measurement target substance. The sensor main body 10 includes an electrode part 11 and a substrate 12. There is no specific designation in the size of the sensor main body 10. However, from the viewpoint of the fixing method and the position to be in contact with the living body, it is preferable to use the one having a width smaller than the diameter of a flexible sheet 21 on a plan view of the position overlapping with the flexible sheet 21 to be described.

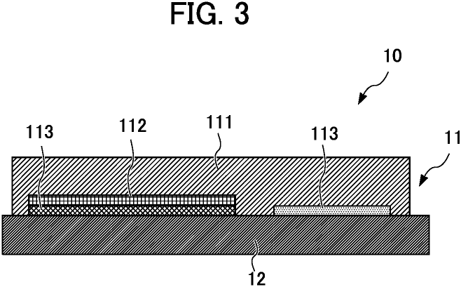

The electrode part 11 is disposed on a contact surface side contacting the living body H. The electrode part 11 absorbs the secretion of the living body H, and measures the electric current value generated by the electrode-detectable substance. As illustrated in FIG. 3, the electrode part 11 includes an absorption layer 111, an enzyme layer 112, and an electrode layer 113.

The absorption layer 111 is a layer capable of absorbing the secretion. The absorption layer 111 is disposed, among the surfaces of the substrate 12 to be described later, on the surface opposing to the living body H. The absorption layer 111 contains a polymer material having a chemically bonded crosslinked structure. The absorption layer 111 covers the end faces of the enzyme layer 112 and the electrode layer 113 to be described later. The absorption layer 111 is configured with a polymer material having a chemically bonded crosslinked structure, for example. The absorption layer 111 is configured to be able to absorb the secretion by contacting the living body H.

The enzyme layer 112 is a layer containing an enzyme that reacts with the absorbed secretion. The enzyme layer 112 contains the enzyme. The enzyme layer 112 is covered by the absorption layer 111. The enzyme layer 112 can contain lactate oxidase, glucose oxidase, alkaline phosphatase, alcohol oxidase, uricase, L-amino acid oxidase, urease, cholesterol oxidase, phosphatase, horseradish peroxidase, and the like, for example. Among those, it is preferable to contain at least one kind selected from lactate oxidase, glucose oxidase, and alcohol oxidase in terms of the fixing method and the position contacting the living body.

The electrode layer 113 is disposed between the substrate 12 and the enzyme layer 112. Furthermore, the electrode layer 113 converts the reaction with the secretion into an electrical signal.

The substrate 12 is disposed on an exposed surface side that is opposite to the contact surface. The substrate 12 supports the electrode part 11. As the substrate 12, for example, it is possible to use thermoplastic elastomers such as a styrenic thermoplastic elastomer, an olefinic thermoplastic elastomer, a diene thermoplastic elastomer, a vinyl chloride thermoplastic elastomer, a urethane thermoplastic elastomer, and a silicone thermoplastic elastomer, thermoplastic resins such as polyethylene, polypropylene, polystyrene, polyethylene terephthalate, cellulose, an acrylonitrile-styrene (AS) resin, glass epoxy, an acrylonitrile-butadiene-styrene (ABS) resin, a methacrylic resin, and vinyl chloride, a polyamide resin, a polyimide resin, and the like. Among those, thermoplastic elastomers are preferable since they are excellent in supportability and flexibility.

The fixing part 20 is used for fixing the sensor main body 10 to the living body H. The fixing part 20 is configured in a size and shape capable of contacting the exposed surface of the substrate 12 and contacting the surface of the living body H. The fixing part 20 includes the flexible sheet 21 and a spacer part 22.

The flexible sheet 21 is attached to the living body H, and fixes the sensor main body 10 to the living body H. The flexible sheet 21 includes an adhesive layer on the surface contacting the living body H.

The spacer part 22 is disposed between the flexible sheet 21 and the exposed surface of the substrate 12. The spacer part 22 contacts the flexible sheet 21 via a contact surface that is opposite to the surface opposing to the substrate 12, for example. Furthermore, the spacer part 22 is configured to have a thickness equal to or thicker than that of the sensor main body 10. The spacer part 22 is configured to have a width larger than that of the substrate 12, for example. The spacer part 22 is configured to be able to transmit, to the substrate 12, the pressing force generated when the flexible sheet 21 is attached to the living body H. The spacer part 22 is configured with a substance having a Young’s modulus capable of maintaining a state where the sensor main body 10 can be kept in continuous contact with the living body H against the strain generated due to the pressing force when the flexible sheet 21 is pressed against the substrate 12, the shape of the sensor main body 10, and the repulsive force from the living body H, for example, and there is no particular specification for the material, but it can be, for example, nonwoven fabric, cloth, cotton, compressed cotton, gauze, other absorbent materials, or combinations thereof.

According to the flexible sheet 21 and the spacer part 22 described above, the flexible sheet 21 has an area larger than the contact surface of the spacer part 22. With such a configuration, the flexible sheet 21 is configured to be able to directly contact the living body H. Furthermore, the flexible sheet 21 includes an adhesive layer on the surface contacting the living body H. Moreover, the flexible sheet 21 also includes an adhesive layer in a part contacting the spacer part 22. The spacer part 22 is attached to the flexible sheet 21 by the adhesive layer. The spacer part 22 is provided along the flexible sheet 21 while being adhered to the flexible sheet 21, for example.

The cover part 30 covers the sensor main body 10 and the fixing part 20 against the living body H, for example. The cover part 30 is an application member for the living body H, such as a band including an elastic body. The cover part 30, by being applied to the living body H, presses the sensor main body 10 and the fixing part 20 against the living body H. The cover part 30 is configured in a width capable of covering the spacer part 22, for example. By having the cover part 30, it is possible to suppress deviation in a state where the sensor main body 10 is kept in continuous contact with the living body.

Next, operational effects of the sensor 1 will be described. First, the sensor main body 10 is brought in contact with the measurement position of the living body H as the measurement target. At this time, the sensor main body 10 is brought in contact with the living body H with the exposed surface of the substrate 12 being exposed. Furthermore, the sensor main body 10 is brought in contact with the position of the living body H that is washed in advance with water, a buffer solution such as saline, alcohol, or the like. Then, the fixing part 20 is attached to the living body H. Specifically, the fixing part 20 is, in a state where the spacer part 22 is being in contact with the exposed surface of the substrate 12, attached to the living body H by bringing the adhesive layer to be in contact with the living body H. Then, the cover part 30 is positioned at the position to cover the fixing part 20 and applied to the living body H. With such a configuration, the sensor 1 is mounted on the living body H.

The sensor 1 according to the embodiment described above can implement the following effects.

(1) A sensor 1 converting an amount of a measurement target substance contained in a secretion from a living body H into an electrical signal, the sensor 1 including: a sensor main body 10 including an electrode part 11 disposed on a contact surface side contacting the living body H, and a substrate 12 disposed on an exposed surface side opposite to the contact surface; and a fixing part 20 that fixes the sensor main body 10 to the living body H, in which the fixing part 20 includes: a flexible sheet 21 that is attached to the living body H, and fixes the sensor main body 10 to the living body H; and a spacer part 22 disposed between the flexible sheet 21 and the exposed surface, the spacer part 22 being in a thickness equal to or thicker than a thickness of the sensor main body 10. With such a configuration, the sensor main body 10 can be appropriately pressed against the surface of the living body H, so that it is possible to suppress deviation while being kept in continuous contact with the living body H.

(2) The flexible sheet 21 includes an adhesive layer on a surface contacting the living body H, and the spacer part 22 is attached to the flexible sheet 21 by the adhesive layer. This makes it possible to maintain the state where the sensor main body 10 is in contact with the living body H, so that it is possible to perform the measurement easily. For example, the operability can be improved when fixing the sensor main body 10 to the living body H after determining the contact position of the sensor main body 10 with respect to the living body H. Furthermore, by decreasing deviation in the contact position of the sensor main body 10 with respect to the living body H after the fixing, it is possible to acquire a living body signal appropriately.

(3) The flexible sheet 21 has an area larger than a contact surface with the spacer part 22. This makes it possible to easily maintain the state of pressing the spacer part 22.

(4) The sensor 1 further includes a cover part 30 that covers the fixing part 20, the cover part 30 pressing the fixing part 20 against the living body H. With such a configuration, deviation of the sensor main body 10 with respect to the living body H can be suppressed more effectively.

(5) The sensor main body 10 contacts a forehead, an upper arm, a back, or a wrist of a human body. This makes it possible to improve the versatility of the sensor 1.

(6) The electrode part 11 includes: an absorption layer 111 capable of absorbing the secretion; an enzyme layer 112 containing an enzyme that reacts with the absorbed secretion; and an electrode layer 113 that converts a reaction with the secretion into an electrical signal. This makes it possible to easily measure the amount of the measurement target substance contained in the secretion.

While each of the preferable embodiments of the sensor according to the present invention is described above, the present invention is not limited to the embodiments and various modifications are possible.

For example, while the cover part 30 is described to cover the entire fixing part 20 in the embodiment above, it is not limited thereto. The cover part 30 may simply need to cover only the spacer main body part.

Furthermore, in the embodiment described above, the shape of the flexible sheet 21 may be changed in accordance with the measurement position of the living body H. For example, when the sensor 1 is applied to the forehead, the flexible sheet 21 may be in a shape with extended long sides on a plan view to fit the forehead. Furthermore, when the sensor 1 is applied to an upper arm, the flexible sheet 21 may be in a shape close to a square or circle on a plan view and may be in a size that does not inhibit the movement of the part where the sensor main body is in contact. Furthermore, like the flexible sheet 21, the spacer part 22 is preferable to be in a shape close to a square or circle on a plan view. Note that the spacer part 22 may simply need to be able to maintain the state where the sensor main body 10 is in contact with the living body H. The spacer part 22 may be configured with an area smaller than that of the sensor main body 10 on a plan view, for example.

### EXPLANATION OF REFERENCE NUMERALS

1 Sensor
H Living body
10 Sensor main body
11 Electrode part
12 Substrate
20 Fixing part
21 Flexible sheet
22 Spacer part
30 Cover part
111 Absorption layer
112 Enzyme layer
113 Electrode layer

1. A sensor converting an amount of a measurement target substance contained in a secretion from a living body into an electrical signal, the sensor comprising:
a sensor main body including an electrode part including an absorption layer capable of absorbing the secretion and having a contact surface side contacting the living body, an enzyme layer containing an enzyme that reacts with the absorbed secretion, and an electrode layer that converts a reaction with the secretion into an electrical signal,
a substrate having an exposed surface opposite to the contact surface; and
a fixing part that fixes the sensor main body to the living body by being brought into contact with the exposed surface, wherein
the fixing part includes:
a flexible sheet that is attached to the living body, and fixes the sensor main body to the living body; and
a spacer part disposed between the flexible sheet and the exposed surface, the spacer part having a thickness equal to or thicker than a thickness of the sensor main body.

2. The sensor according to claim 1, wherein the flexible sheet includes an adhesive layer on a surface contacting the living body, and
the spacer part is attached to the flexible sheet by the adhesive layer.

3. The sensor according to claim 1, wherein the flexible sheet has an area larger than a contact surface with the spacer part.

4. The sensor according to claim 1, further comprising a cover part that covers the fixing part, the cover part pressing the fixing part against the living body.

5. The sensor according to claim 1, wherein the sensor main body contacts a forehead, an upper arm, a back, or a wrist of a human body.

6. (canceled)

7. The sensor according to claim 1, wherein the enzyme layer contains at least one kind selected from lactate oxidase, glucose oxidase, and alcohol oxidase.

8. The sensor according to claim 1, wherein the absorption layer contains a polymer material having a chemically bonded crosslinked structure, the absorption layer covering end faces of the enzyme layer and the electrode layer.

9. The sensor according to claim 4, wherein the sensor main body contacts a forehead, an upper arm, a back, or a wrist of a human body.
